(19) United States
(12) Patent Application Publication
(10) Pub. No.: US 2016/0340674 A1
(43) Pub. Date: Nov. 24, 2016

(54) Compositions and Methods to Inhibit EZH2 for the Treatment of Cardiovascular Diseases

(71) Applicant: UNIVERSITY OF ROCHESTER, Rochester, NY (US)

(72) Inventor: Zheng-Gen JIN, Rochester, NY (US)

(21) Appl. No.: 15/114,947

(22) PCT Filed: Feb. 9, 2015
(86) PCT No.: PCT/US2015/014989

§ 371 Date: Jul. 28, 2016

Related U.S. Application Data
(60) Provisional application No. 61/937,672, filed on Feb. 10, 2014.

(51) Int. Cl. C12N 15/113; A61K 31/713; A61K 45/06; A61K 31/496

(52) U.S. Cl. CPC C12N 15/113; A61K 31/496; A61K 31/713; A61K 45/06; C12N 2310/14

(57) ABSTRACT

The present invention relates to compositions and methods for treatment and/or prevention of a cardiovascular disease. In one embodiment, the invention provides compositions and methods for decreasing one or more of the level, production, and activity of EZH2.

### CROSS-REFERENCE TO RELATED APPLICATIONS

This application claims priority to U.S. Provisional Application Ser. No. 61/937,672, filed Feb. 10, 2014, the content of which is incorporated by reference herein in its entirety.

### STATEMENT REGARDING FEDERALLY SPONSORED RESEARCH OR DEVELOPMENT

This invention was made with government support under 2R0109502-01A1 awarded by the National Institutes of Health (NIH). The government has certain rights in the invention.

### BACKGROUND OF THE INVENTION

Cardiovascular disease (CVD) is the single largest killer of adults in North America (Heart Disease and Stroke Statistics—2008 Update. A Report from the American Heart Association Statistics Committee and Stroke Statistics Subcommittee). CVD includes diseases caused by atherosclerosis, such as coronary heart disease (CHD), ischemic stroke and peripheral arterial disease (PAD). Atherosclerosis is a disease of the arterial blood vessel walls, resulting from endothelial cell dysfunction, high plasma cholesterol levels, foam cell formation and local inflammation. CHD is caused by the development and progression of atherosclerotic lesions in coronary arteries which results in acute coronary syndrome (ACS; i.e. unstable angina & myocardial infarction). In 2005 there were estimated to be 772,000 ACS patients in the U.S. (Heart Disease and Stroke Statistics—2008 Update. A Report from the American Heart Association Statistics Committee and Stroke Statistics Subcommittee). Approximately 1 in 5 deaths in 2004 were due to CHD, with a total U.S. and Canadian mortality of over 500,000 individuals. It is estimated that over 100 million North Americans have high blood cholesterol levels placing them in a border-line high risk, or high risk category of developing CHD. The total U.S. prevalence of ischemic stroke in 2005 was approximately 4.6 million and the annual incidence for both first time and recurrent attacks was around 780,000 (Abramson and Huckell, Can J Cardiol 2005 21(2): 997-1006). PAD is characterized by restricted blood flow to the extremities (e.g. legs, feet) resulting in cramping and in severe cases loss of the limb. According to the Society of Interventional Radiology, people over the age of 50 who smoke or have diabetes are at increased risk of developing PAD. Sixteen percent of individuals in North America have PAD. There are about 30 million people worldwide with PAD, half of which are asymptomatic. The estimated prevalence for PAD is 4% of the population over the age of 40 (Abramson and Huckell, Can J Cardiol 2005 21(2):997-1006). The survival rate for severe symptomatic patients is approximately 25% (Abramson and Huckell Can J Cardiol 2005 21(2): 997-1006).

Currently there are no satisfactory modes of therapy for the prevention and/or treatment of cardiovascular and cardiovascular-related diseases and there is a need therefore to develop new therapies for this purpose. Therefore, there remains an unmet need for compositions and methods of treating cardiovascular and cardiovascular-related diseases. The present invention satisfies these unmet needs.

### SUMMARY OF THE INVENTION

In one embodiment, the invention provides a method for treating a cardiovascular disease in a subject comprising administering to a subject an effective amount of a compound selected from (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide, N-((4,6-dimethyl-2-oxo-1,2-dihydropyridin-3-yl)methyl)-1-isopropyl-3-methyl-6-(6-(4-methylpiperazin-1-yl)pyridin-3-yl)-1H-indole-4-carboxamide, N-((4,6-dimethyl-2-oxo-1,2-dihydropyridin-3-yl)methyl)-6-(6-(hydroxymethyl)pyridin-3-yl)-1-isopropyl-3-methyl-1H-indole-4-carboxamide, N-((4,6-dimethyl-2-oxo-1,2-dihydropyridin-3-yl)methyl)-1-isopropyl-3-methyl-6-(oxetan-3-yl)-1H-indole-4-carboxamide, N-((4,6-dimethyl-2-oxo-1,2-dihydropyridin-3-yl)methyl)-1-isopropyl-3-methyl-6-(4-methylpiperazine-1-carboxamido)-1H-indole-4-carboxamide, N-((4,6-dimethyl-2-oxo-1,2-dihydropyridin-3-yl)methyl)-6-((3-(dimethylamino)propyl)thio)-1-isopropyl-3-methyl-1H-indole-4-carboxamide, N-((4,6-dimethyl-2-oxo-1,2-dihydropyridin-3-yl)methyl)-6-((3-(dimethylamino)propyl)thio)-1-isopropyl-3-methyl-1H-indole-4-carboxamide, 6-(3-hydroxy-3-methylbut-1-yn-1-yl)-1-isopropyl-3-methyl-N-((6-methyl-2-oxo-4-propyl-1,2-dihydropyridin-3-yl)methyl)-1H-indole-4-carboxamide, 6-(cyclopropylethynyl)-1-isopropyl-3-methyl-N-((6-methyl-2-oxo-4-propyl-1,2-dihydropyridin-3-yl)methyl)-1H-indole-4-carboxamide, 1-cyclopentyl-N-((4,6-dimethyl-2-oxo-1,2-dihydropyridin-3-yl)methyl)-6-(morpholinomethyl)-1H-indazole-4-carboxamide, N-((4,6-dimethyl-2-oxo-1,2-dihydropyridin-3-yl)methyl)-3-(ethyl(tetrahydro-2H-pyran-4-yl)amino)-2-methyl-5-(morpholinomethyl)benzamide, (1S,2R,5R)-5-(4-amino-1H-imidazo [4,5-c]pyridin-1-yl)-3-(hydroxymethyl)cyclopent-3-ene-1,2-diol, or 1-isopropyl-N-((6-methyl-2-oxo-4-propyl-1,2-dihydropyridin-3-yl)methyl)-6-(6-(4-methylpiperazin-1-yl)pyridin-3-yl)-1H-indazole-4-carboxamide.

In one embodiment, the compound is (S)-1-(sec-butyl)-N-((4,6-dimethyl-2-oxo-1, 2-dihydropyridin-3-yl)methyl)-3-methyl-6-(6-(piperazin-1-yl)pyridin-3-yl)-1H-indole-4-carboxamide.

In one embodiment, the compound is 1-isopropyl-N-((6-methyl-2-oxo-4-propyl-1,2-dihydropyridin-3-yl)methyl)-6-(6-(4-methylpiperazin-1-yl)pyridin-3-yl)-1H-indazole-4-carboxamide.

In one embodiment, the invention provides a method for treating a cardiovascular disease in a subject, the method comprising administering to a subject in need thereof an effective amount of a siRNA that forms a complex with a region in EZH2 mRNA.

In one embodiment, the siRNA comprises a sequence complementary to a region in EZH2 mRNA.

In one embodiment, the siRNA comprises a sequence that is complementary to a region having a sequence selected from SEQ ID NOs:1, 2 or 3.

In one embodiment, the cardiovascular disease is selected from the group consisting of coronary artery disease, hypertension, heart failure, diabetic cardiovascular complications, atherosclerosis, coronary heart disease, angina, stroke, ischemia and myocardial infarction, and any combination thereof.

In one embodiment, the method of treating a cardiovascular disease in a subject of the invention further comprises administering a second agent to the subject. In one embodiment, the second agent is selected from the group consisting of ACE inhibitors, ARB's, adrenergic blockers, adrenergic agonists, agents for pheochromocytoma, anti-arrhythmics, antiplatelet agents, anticoagulants, antihypertensives, antilipemic agents, antidiabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, renin inhibitors, vasodilators, vasopressors, AGE crosslink breakers, AGE formation inhibitors, and any combinations thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A and 1B, is a series of images demonstrating that inhibition of EZH2 by GSK126 increases the levels of KLF2 and eNOS mRNA in endothelial cells in a time-dependent manner. FIG. 1A shows RT-PCR data. FIG. 1B shows q-PCR data.

FIG. 2, comprising FIGS. 2A and 2B, is a series of images demonstrating that GSK126 increases the levels of KLF2 (FIG. 2A) and eNOS (FIG. 2B) mRNA in endothelial cells in a dose-dependent manner.

FIG. 3, comprising FIGS. 3A through 3E, is a series of images demonstrating that knockdown of EZH2 by small interference RNA (siRNA) increases expression of atheroprotective genes KLF2 and eNOS in endothelial cells. Human umbilical vein endothelial cells (HUVECs) were treated with control siRNA (siCtrl, 100 nM) and siRNA targeting EZH2 (siEZH2, 100 nM) for 48 hrs. The cell lysates were collected for gene expression analysis by Q-PCR for eNOS (FIG. 3A), KLF2 mRNA (FIG. 3B) and GAPDH mRNA as an internal control. Cell lysates were also analyzed for protein levels of EZH2, eNOS and tubulin (internal control) with Western blots (FIG. 3C). HUVECs were exposed to laminar flow (L-flow, 12 dyne/cm2) for 24 hours. The cell lysates were analyzed for protein levels of EZH2, eNOS and tubulin (internal control) with Western blots (FIG. 3D). HUVECs were treated with control siRNA and EZH2 siRNA for 48 hours and then exposed to laminar flow for 24 hours. The levels of EZH2, eNOS and tubulin were analyzed (FIG. 3E). Three independent experiments were performed and representative images were shown. * p<0.05.

FIG. 4, comprising FIGS. 4A and 4B is a series of images showing haploinsufficiency of EZH2 attenuates atherosclerotic lesion size in ApoE⁴⁻mice. (FIG. 4A) Atherosclerosis in the arterial tree was evaluated by Oil Red 0 staining. (FIG. 4B) Quantification of Oil Red O-positive areas in en face aorta by Image-Pro Plus software. n=4 for ApoE^{-/-}; EZH2^{+/+} control group, n=5 for ApoE; EZH2⁺¹ group. *P<0.05, compared to control group.

### DETAILED DESCRIPTION

The present invention is partly based upon the discovery that inhibition of EZH2 results in stimulating vascular endothelial cell gene expression including Kruppel-like factors 2 (KLF2) and endothelial nitric oxide synthase (eNOS). Accordingly, the invention provides compositions and methods of inhibiting EZH2 as a therapy to treat cardiovascular diseases. Non-limiting examples of a cardiovascular disease include but is not limited to coronary artery disease, hypertension, heart failure, diabetic cardiovascular complications, and the like.

In one embodiment, the present invention is directed to methods and compositions for treatment, inhibition, prevention, or reduction of a cardiovascular disease. In one embodiment, the invention provides compositions and methods for modulating one or more of the level, production, and activity of EZH2.

Accordingly, the invention provides inhibitors (e.g., antagonists) of EZH2. In one embodiment, the inhibitor of EZH2 includes but is not limited to an antibody or a fragment thereof, a peptide, a nucleic acid, a ribozyme, an aptamer, a small molecule, a chemical compound, and the like.

In one embodiment, the present invention comprises a method for decreasing one or more of the level, production, and activity of EZH2, comprising administering to a subject an effective amount of a composition comprising an inhibitor of EZH2. In an embodiment of the present invention, the composition decreases the transcription of EZH2 or translation of EZH2 mRNA. In another embodiment of the present invention, the composition inhibits the activity of EZH2 activity.

Another aspect of the present invention comprises a pharmaceutical composition comprising an inhibitor of EZH2. In one embodiment, the composition of the invention can be used in combination with another therapeutic agent.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the term “acute coronary syndrome”, (ACS) refers to any group of symptoms attributed to obstruction of the coronary arteries. The most common symptom prompting diagnosis of ACS is chest pain, often radiating of the left arm or angle of the jaw, pressure-like in character, and associated with nausea and sweating.

As used herein, the term “acute decompensated heart failure”, (ADHF) refers to a worsening of the symptoms, typically shortness of breath (dyspnea), edema and fatigue, in a patient with existing heart disease. ADHF is a common and potentially serious cause of acute respiratory distress.

As used herein, the term “atherosclerosis” refers to the progressive accumulation of smooth muscle cells, immune cells (e.g., lymphocytes, macrophages, or monocytes), lipid products (e.g., lipoproteins, or cholesterol), cellular waste products, calcium, or other substances within the inner lining of an artery, resulting in the narrowing or obstruction of the blood vessel and the development of atherosclerosis-associated diseases. Atherosclerosis is typically manifested within large and medium-sized arteries, and is often characterized by a state of chronic inflammation within the arteries.

As used herein, the term “atherosclerosis-associated disease” refers to any disorder that is caused by or is associated with atherosclerosis. Typically, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis).

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L}, or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L}, and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, “aptamer” refers to a small molecule that can bind specifically to another molecule. Aptamers are typically either polynucleotide- or peptide-based molecules. A polynucleotide aptamer is a DNA or RNA molecule that adopts a highly specific three-dimensional conformation designed to have appropriate binding affinities and specificities towards specific target molecules, such as peptides, proteins, drugs, vitamins, among other organic and inorganic molecules. Such polynucleotide aptamers can be selected from a vast population of random sequences through the use of systematic evolution of ligands by exponential enrichment. A peptide aptamer is typically a loop of about 10 to about 20 amino acids attached to a protein scaffold that binds to specific ligands. Peptide aptamers may be identified and isolated from combinatorial libraries, using methods such as the yeast two-hybrid system.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, the term “cardiovascular disease” or “CVD,” generally refers to heart and blood vessel diseases, including atherosclerosis, coronary heart disease, cerebrovascular disease, and peripheral vascular disease. Cardiovascular disorders are acute manifestations of CVD and include myocardial infarction, stroke, angina pectoris, transient ischemic attacks, and congestive heart failure.

Cardiovascular disease, including atherosclerosis, usually results from the build-up of cholesterol, inflammatory cells, extracellular matrix and plaque. The term “cardiovascular disease” also includes indications caused by oxidative stress by reactive oxygen species, and includes but is not limited to angina pectoris, coronary heart disease, hypertension, endothelial dysfunction, atherosclerosis and the like.

The term “cardiac dysfunction” refers to a pathological decline in cardiac performance. Cardiac dysfunction may be manifested through one or more parameters or indicies including changes to stroke volume, ejection fraction, end diastolic fraction, stroke work, arterial elastance (defined as the ratio of left ventricular (LV) end-systolic pressure and stroke volume), or an increase in heart weight to body weight ratio. Unless otherwise noted, cardiac dysfunctions encompass any cardiac disorders or aberrant conditions that are associated with or induced by the various cardiomyopathies, cardiomyocyte hypertrophy, cardiac fibrosis, or other cardiac injuries described herein. Specific examples of cardiac dysfunction include cardiac remodeling, cardiac hypertrophy, and heart failure.

As used herein, the terms “congestive heart failure, (CHF)” “chronic heart failure,” “acute heart failure,” and “heart failure” are used interchangeably, and refer to any condition in which the heart is unable to pump blood at an adequate rate or to do so only in the presence of increased left ventricular filling pressures. When the heart is unable to adequately pump blood to the rest of the body at normal filling left ventricular pressures, blood can back up into the lungs, causing the lungs to become congested with fluid. Typical symptoms of heart failure include shortness of breath (dyspnea), fatigue, weakness, difficulty breathing when lying flat, and swelling of the legs, ankles or abdomen (edema). Causes of heart failure are related to various disorders including coronary artery disease, systemic hypertension, cardiomyopathy or myocarditis, congenital heart disease, abnormal heart valves or valvular heart disease, severe lung disease, diabetes, severe anemia hyperthyroidism, arrhythmia or dysrhythmia and myocardial infarction. Heart failure can occur in the presence of a normal (>50%) or a reduced (<50%) left ventricular ejection fraction. There is increased recognition that these two conditions represent two different disease states, rather than a continuum (Borlaug B A, Redfield M M. Circulation. 2011 May 10; 123(18):2006-13).

As used herein, the term “coronary heart disease” or “CHD” refers to atherosclerosis in the arteries of the heart causing a heart attack or other clinical manifestation such as unstable angina.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity or frequency of at least one sign or symptom of the disease or disorder experienced by a patient is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, a “recombinant cell” is a host cell that comprises a recombinant polynucleotide.

“Sample” or “biological sample” as used herein means a biological material from a subject, including but is not limited to organ, tissue, exosome, blood, plasma, saliva, urine and other body fluid. A sample can be any source of material obtained from a subject.

By the term “specifically binds,” as used herein, is meant a molecule, such as an antibody, which recognizes and binds to another molecule or feature, but does not substantially recognize or bind other molecules or features in a sample.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The invention is based partly on the discovery that inhibition of EZH2 stimulated gene expression of genes that plays important roles in preventing or treating endothelial dysfunction and vascular inflammation including but not limited to Kruppel-like factors 2 (KLF2) and endothelial nitric oxide synthase (eNOS). Therefore, inhibition of EZH2 provides a new strategy to prevent or treat cardiovascular diseases.

The present invention relates generally to compositions and methods for inhibiting EZH2 to treat cardiovascular diseases. In one embodiment, the present invention is directed to methods and compositions for treatment, inhibition, prevention, or reduction of a cardiovascular disease using an inhibitor of EZH2.

In one embodiment, the present invention provides a composition for treating a cardiovascular disease in a subject, wherein the composition comprises an inhibitor of EZH2.

### Compositions

In one embodiment, the invention provides an inhibitor of EZH2. In various embodiments, the present invention includes compositions for inhibiting the level or activity of EZH2 in a subject, a cell, a tissue, or an organ in need thereof. In various embodiments, the compositions of the invention inhibits the amount of polypeptide of EZH2, the amount of mRNA of EZH2, the amount of activity of EZH2, or a combination thereof.

The compositions of the invention include compositions for treating or preventing cardiovascular diseases. In various embodiments, the composition for treating a cardiovascular disease comprises an inhibitor of EZH2. In one embodiment, the inhibitor of the invention decreases the amount of EZH2 polypeptide, the amount of EZH2 mRNA, the amount of EZH2 activity, or a combination thereof.

It will be understood by one skilled in the art, based upon the disclosure provided herein, that a decrease in the level of EZH2 encompasses the decrease in the expression, including DNA transcription, mRNA translation, mRNA stability, protein stability or any all of their combinations. The skilled artisan will also appreciate, once armed with the teachings of the present invention, that a decrease in the level of EZH2 includes a decrease in the activity of EZH2. Thus, decrease in the level or activity of EZH2 includes, but is not limited to, decreasing the amount of polypeptide of EZH2, and decreasing transcription, translation, or both, of a nucleic acid encoding EZH2; and it also includes decreasing any activity of EZH2 as well.

In one embodiment, the invention provides a generic concept for inhibiting EZH2 therapy to treat cardiovascular diseases. In one embodiment, the composition of the invention comprises an inhibitor of EZH2. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide, an aptamer and a small molecule.

Nucleic Acid Inhibitors

One skilled in the art will appreciate, based on the disclosure provided herein, that one way to decrease the mRNA and/or protein levels of EZH2 in a cell is by reducing or inhibiting expression of the nucleic acid encoding EZH2. Thus, the protein level of EZH2 in a cell can also be decreased using a molecule or compound that inhibits or reduces gene expression such as, for example, siRNA, an antisense molecule or a ribozyme. However, the invention should not be limited to these examples.

In one embodiment, siRNA is used to decrease the level of EZH2. In one embodiment, siRNA is used to treat cardiovascular diseases. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of EZH2 at the protein level using RNAi technology.

In one embodiment, the siRNA's are the ones hybridized to the following regions of EZH2 mRNA:

(SEQ ID NO: 1)
5′ AGGAUACAGACAGUGAUAGGGAAGC 3′
(SEQ ID NO: 2)
5′ GGCACUUACUAUGACAAUUUCUGUG 3′
(SEQ ID NO: 3)
5′ GCUCUAGACAACAAACCUUGUGGAC 3′

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)). These modifications can be applied to any nucleic acid molecule of the invention.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In other related aspects, the invention includes an isolated nucleic acid encoding an inhibitor, wherein an inhibitor such as an siRNA or antisense molecule, inhibits EZH2, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York) and as described elsewhere herein. In another aspect of the invention, EZH2 or a regulator thereof, can be inhibited by way of inactivating and/or sequestering one or more of EZH2, or a regulator thereof. As such, inhibiting the effects of EZH2 can be accomplished by using a transdominant negative mutant.

In another aspect, the invention includes a vector comprising an siRNA or antisense polynucleotide. Preferably, the siRNA or antisense polynucleotide is capable of inhibiting the expression of EZH2. The incorporation of a desired polynucleotide into a vector and the choice of vectors is well-known in the art as described in, for example, Sambrook et al., supra.

The siRNA or antisense polynucleotide can be cloned into a number of types of vectors as described elsewhere herein. For expression of the siRNA or antisense polynucleotide, at least one module in each promoter functions to position the start site for RNA synthesis.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit EZH2. The antisense expressing vector is used to transfect a mammalian cell or the mammal itself, thereby causing reduced endogenous expression of EZH2.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit EZH2. Ribozymes useful for inhibiting the expression of a target molecule may be designed by incorporating target sequences into the basic ribozyme structure which are complementary, for example, to the mRNA sequence of EZH2 of the present invention. Ribozymes targeting EZH2 may be synthesized using commercially available reagents (Applied Biosystems, Inc., Foster City, Calif.) or they may be genetically expressed from DNA encoding them.

MicroRNA

MicroRNA is a small non-coding RNA which inhibits gene expression at a control step after transcription. Generally, a microRNA is composed of 18 to 25 nucleotides on average and forms a hairpin structure. It complementarily binds to a 3′-UTR portion of the sequence of a target gene to inhibit mRNA from decomposing or translating to a protein, and it has been known that at least about 5000 human genes are targets of microRNAs. Functions of microRNAs in vivo can be various, and for instance, include cell differentiation and proliferation, control of developmental stages and metabolism, angiogenesis, and apoptosis, depending on what type of target gene is eventually controlled.

In one embodiment, the invention includes the use of microRNAs to target EZH2. Examples of microRNAs that target EZH2 includes but is not limited to miR-101 (Cancer Res. 2009 Mar. 15; 69(6):2623-9), miR-26a (J Biol Chem. 2008 Apr. 11; 283(15):9836-43), miR-214 (Mol Cell. 2009 Oct. 9; 36(1):61-74), miR-137 (J Cell Biol. 2010 Apr. 5; 189(1):127-41), miR-138 (Eur J Neurosci. 2011 January; 33(2):224-35), miR-98 (Cell Death Dis. 2010 Oct. 21; 1:e85), Let-7a (Oncol Rep. 2012 December; 28(6):2101-6), Let-7c (Stem Cell Res. 2013 Nov. 28; 12(2):323-337), miR-31 (Oncotarget. 2012 September; 3(9):1011-25), miR-708 (Oncol Rep. 2013 August; 30(2):870-6), miR-144 (FEBS J. 2013 September; 280(18):4531-8), and the likes.

Another aspect of the invention relates to a therapeutic agent characterized by its ability to modulate the level of one or more microRNA that targets EZH2. Therefore, in one embodiment, the invention includes modulating the level, activity and/or expression of at least one of miR-101, miR-26a, miR-214, miR-137, miR-138, miR-98, Let-7a, Let-7c, miR-31, miR-708, miR-144 in order to inhibit EZH2.

Aptamers

In one embodiment, the composition comprises an aptamer, including for example a protein aptamer or a polynucleotidal aptamer. In one embodiment, the aptamer inhibits the expression, activity, or both of EZH2.

In one embodiment, an apatmer is a nucleic acid or oligonucleotide molecule that binds to a specific molecular target, such as EZH2. In one embodiment, aptamers are obtained from an in vitro evolutionary process known as SELEX (Systematic Evolution of Ligands by EXponential Enrichment), which selects target-specific aptamer sequences from combinatorial libraries of single stranded oligonucleotide templates comprising randomized sequences. In some embodiments, aptamer compositions are double-stranded or single-stranded, and in various embodiments include deoxyribonucleotides, ribonucleotides, nucleotide derivatives, or other nucleotide-like molecules. In some embodiments, the nucleotide components of an aptamer include modified or non-natural nucleotides, for example nucleotides that have modified sugar groups (e.g., the 2′-OH group of a ribonucleotide is replaced by 2′-F or 2′-NH₂), which in some instances, improves a desired property, e.g., resistance to nucleases or longer lifetime in blood.

In some instances, individual aptamers having the same nucleotide sequence differ in their secondary structure. In some embodiments, the aptamers of the invention are conjugated to other molecules, e.g., a high molecular weight carrier to slow clearance of the aptamer from the circulatory system. In some instances, aptamers are specifically cross-linked to their cognate ligands, e.g., by photo-activation of a cross-linker. (Brody, E. N. and L. Gold (2000) J. Biotechnol. 74:5-13).

A method for the in vitro evolution of nucleic acid molecules with high affinity binding to target molecules is known to those of skill in the art and is described in U.S. Pat. No. 5,270,163. The method, known as SELEX (Selective Evolution of Ligands by EXponential Enrichment) involves selection from a mixture of candidate oligonucleotides from a library comprising a large sequence variations (e.g. about 10¹⁵) and step-wise iterations of binding, partitioning and amplification, using the same general selection theme, to achieve virtually any desired criterion of binding affinity and selectivity.

Starting from a mixture of nucleic acids, preferably comprising a segment of randomized sequence, the SELEX method includes the steps of contacting the mixture with the desired target, partitioning unbound nucleic acids from those nucleic acids which have bound to the target molecule, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield high affinity nucleic acid ligands to the target molecule.

Peptide Inhibitors

In other related aspects, the invention includes an isolated peptide inhibitor that inhibits EZH2. For example, in one embodiment, the peptide inhibitor of the invention inhibits EZH2 directly by binding to EZH2 thereby preventing the normal functional activity of EZH2. In another embodiment, the peptide inhibitor of the invention inhibits EZH2 by competing with endogenous EZH2. In yet another embodiment, the peptide inhibitor of the invention inhibits the activity of EZH2 by acting as a transdominant negative mutant.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

A peptide inhibitor of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide inhibitor.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

(a) Tags

In a particular embodiment of the invention, the polypeptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

(b) Leader and Secretory Sequences

Accordingly, the polypeptides of the invention can be fused to another polypeptide or tag, such as a leader or secretory sequence or a sequence which is employed for purification or for detection. In a particular embodiment, the polypeptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the polypeptide sequence, thus permitting the removal of the binding tag after incubation with this specific enzyme and thus facilitating the purification of the corresponding protein of interest.

(c) Targeting Sequences

The invention also relates to a chimeric peptide comprising a peptide inhibitor described herein, fused to a targeting domain capable of directing the chimeric peptide to a desired cellular component or cell type or tissue. The chimeric peptide may also contain additional amino acid sequences or domains. The chimeric peptide are recombinant in the sense that the various components are from different sources, and as such are not found together in nature (i.e., are heterologous).

The targeting domain can be a membrane spanning domain, a membrane binding domain, or a sequence directing the peptide to associate with for example vesicles or with the nucleus. The targeting domain can target a peptide inhibitor to a particular cell type or tissue. For example, the targeting domain can be a cell surface ligand or an antibody against cell surface antigens of a target tissue (e.g., skin or melanocyte). A targeting domain may target a peptide inhibitor to a cellular component.

(d) Intracellular Targeting

Combined with certain formulations, such peptides can be effective intracellular agents. However, in order to increase the efficacy of such peptides, the peptide inhibitor can be provided as a fusion or chimeric peptide comprising a second peptide which promotes “transcytosis”, e.g., uptake of the peptide by cells. To illustrate, the peptide inhibitor of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the peptide inhibitor can be provided a fusion polypeptide with all or a portion of the antenopedia III protein.

To further illustrate, the peptide inhibitor can be provided as a chimeric peptide which includes a heterologous peptide sequence (“internalizing peptide”) which drives the translocation of an extracellular form of a peptide inhibitor across a cell membrane in order to facilitate intracellular localization of the peptide inhibitor. In this regard, the therapeutic peptide inhibitor is one which is active intracellularly. The internalizing peptide, by itself, is capable of crossing a cellular membrane by, e.g., transcytosis, at a relatively high rate. The internalizing peptide is conjugated, e.g., as a fusion protein, to the peptide inhibitor. The resulting chimeric peptide is transported into cells at a higher rate relative to the activator polypeptide alone to thereby provide a means for enhancing its introduction into cells to which it is applied.

In one embodiment, the composition comprises a peptidomimetic inhibitor of EZH2. Peptidomimetics are compounds based on, or derived from, peptides and proteins. The peptidomimetics of the present invention typically can be obtained by structural modification of known EZH2 sequences or sequences that interact with EZH2, using unnatural amino acids, conformational restraints, isosteric replacement, and the like. The peptidomimetics constitute the continum of structural space between peptides and non-peptide synthetic structures.

Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in Peptides: Chemistry and Biology, G. R. Marshall ed., ESCOM Publisher: Leiden, Netherlands, 1988)

In addition to a variety of side chain replacements which can be carried out to generate peptidomimetics, the present invention contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

In one embodiment, the inhibitor of the invention comprises a mimetope. Examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to EZH2. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling, the predicted mimetope structures can then be produced by, for example, chemical synthesis, recombinant DNA technology, or by isolating a mimetope from a natural source (e.g., plants, animals, bacteria and fungi).

A peptide or peptidomimetic inhibitor of the invention may be synthesized by conventional techniques. For example, the peptide or peptidomimetic inhibitor may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

N-terminal or C-terminal fusion proteins comprising a peptide or peptidomimetic inhibitor of the invention conjugated with other molecules may be prepared by fusing, through recombinant techniques, the N-terminal or C-terminal of the peptide or peptidomimetic inhibitor, and the sequence of a selected protein or selectable marker with a desired biological function. The resultant fusion proteins contain the peptide inhibitor, or chimeric protein fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptide or peptidomimetic inhibitor of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Prior to its use as an inhibitor, a peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Antibody Inhibitors

In another aspect of the invention, EZH2 can be inhibited by way of inactivating and/or sequestering EZH2. As such, inhibiting the effects of EZH2 can be accomplished by using a transdominant negative mutant. Alternatively an antibody specific for EZH2 (e.g., an antagonist to EZH2) may be used. In one embodiment, the antagonist is a protein and/or compound having the desirable property of interacting with a binding partner of EZH2 and thereby competing with the corresponding protein. In another embodiment, the antagonist is a protein and/or compound having the desirable property of interacting with EZH2 and thereby sequestering EZH2.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic affinity cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art. In one embodiment, the EZH2 inhibitor is a small molecule compound having structures in Table 1.

TABLE 1
Small Molecule Compounds as EZH2 inhibitors

| Name | Structure |
|---|---|
| (S)-1-(sec-butyl)-N-((4,6-dimethyl- 2-oxo-1,2-dihydropyridin-3- yl)methyl)-3-methyl-6-(6-(piperazin- 1-yl)pyridin-3-yl)-1H-indole-4- carboxamide (GSK126) | 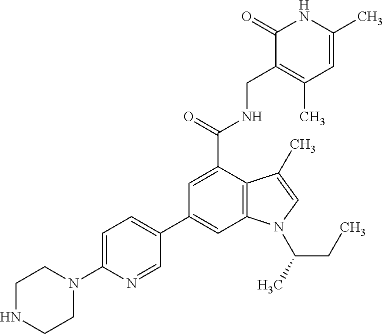 [SMILES: CC[C@H](C)n1cc(C)c2c(C(=O)NCc3c(C)cc(C)[nH]c3=O)cc(-c3ccc(N4CCNCC4)nc3)cc21] |
| N-((4,6-dimethyl-2-oxo-1,2- dihydropyridin-3-yl)methyl)-1- isopropyl-3-methyl-6-(6-(4- methylpiperazin-1-yl)pyridin-3-yl)- 1H-indole-4-carboxamide | 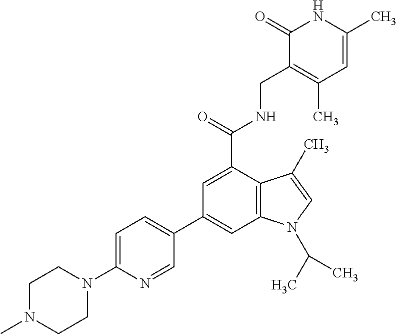 |
| N-((4,6-dimethyl-2-oxo-1,2- dihydropyridin-3-yl)methyl)-6-(6- (hydroxymethyl)pyridin-3-yl)-1- isopropyl-3-methyl-1H-indole-4- carboxamide | 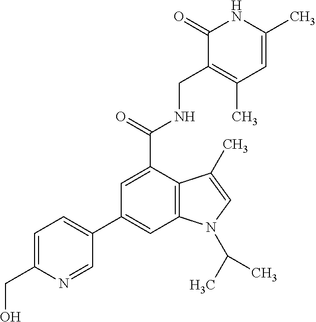 [SMILES: Cc1cc(C)c(CNC(=O)c2cc(-c3ccc(CO)nc3)cc3c2c(C)cn3C(C)C)c(=O)[nH]1] |
| N-((4,6-dimethyl-2-oxo-1,2- dihydropyridin-3-yl)methyl)-1- isopropyl-3-methyl-6-(oxetan-3-yl)- 1H-indole-4-carboxamide | 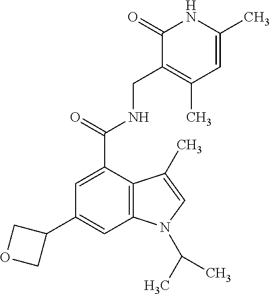 [SMILES: Cc1cc(C)c(CNC(=O)c2cc(C3COC3)cc3c2c(C)cn3C(C)C)c(=O)[nH]1] |
| N-((4,6-dimethyl-2-oxo-1,2- dihydropyridin-3-yl)methyl)-1- isopropyl-3-methyl-6-(4- methylpiperazine-1-carboxamido)- 1H-indole-4-carboxamide | 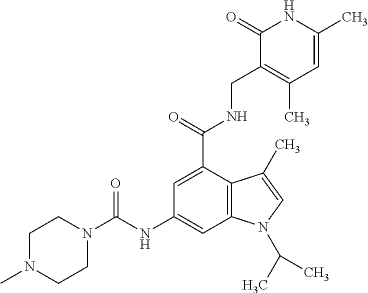 |
| N-((4,6-dimethyl-2-oxo-1,2- dihydropyridin-3-yl)methyl)-6-((3- (dimethylamino)propyl)thio)-1- isopropyl-3-methyl-1H-indole-4- carboxamide | 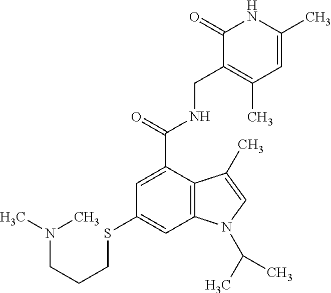 |
| N-((4,6-dimethyl-2-oxo-1,2- dihydropyridin-3-yl)methyl)-6-(3- hydroxy-3-methylbut-1-yn-1-yl)-1- isopropyl-3-methyl-1H-indole-4- carboxamide | 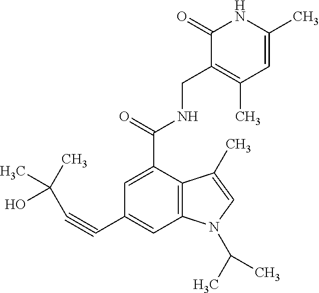 [SMILES: Cc1cc(C)c(CNC(=O)c2cc(C#CC(C)(C)O)cc3c2c(C)cn3C(C)C)c(=O)[nH]1] |
| 6-(3-hydroxy-3-methylbut-1-yn-1- yl)-1-isopropyl-3-methyl-N-((6- methyl-2-oxo-4-propyl-1,2- dihydropyridin-3-yl)methyl)-1H- indole-4-carboxamide | 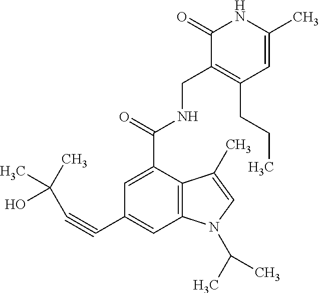 [SMILES: CCCc1cc(C)[nH]c(=O)c1CNC(=O)c1cc(C#CC(C)(C)O)cc2c1c(C)cn2C(C)C] |
| 6-(cyclopropylethynyl)-1-isopropyl- 3-methyl-N-((6-methyl-2-oxo-4- propyl-1,2-dihydropyridin-3- yl)methyl)-1H-indole-4- carboxamide | 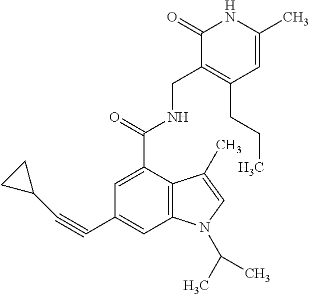 [SMILES: CCCc1cc(C)[nH]c(=O)c1CNC(=O)c1cc(C#CC2CC2)cc2c1c(C)cn2C(C)C] |
| 1-cyclopentyl-N-((4,6-dimethyl-2- oxo-1,2-dihydropyridin-3- yl)methyl)-6-(morpholinomethyl)- 1H-indazole-4-carboxamide | 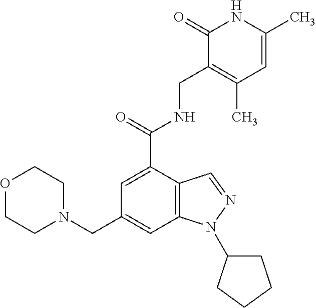 [SMILES: Cc1cc(C)c(CNC(=O)c2cc(CN3CCOCC3)cc3c2cnn3C2CCCC2)c(=O)[nH]1] |
| N-((4,6-dimethyl-2-oxo-1,2- dihydropyridin-3-yl)methyl)-3- (ethyl(tetrahydro-2H-pyran-4- yl)amino)-2-methyl-5- (morpholinomethyl)benzamide | 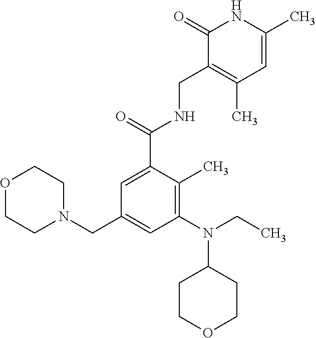 [SMILES: CCN(c1cc(CN2CCOCC2)cc(C(=O)NCc2c(C)cc(C)[nH]c2=O)c1C)C1CCOCC1] |
| (1S,2R,5R)-5-(4-amino-1H- imidazo[4,5-c]pyridin-1-yl)-3- (hydroxymethyl)cyclopent-3-ene- 1,2-diol | 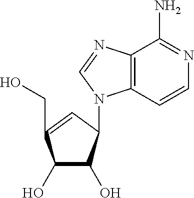 |
| 1-isopropyl-N-((6-methyl-2-oxo-4- propyl-1,2-dihydropyridin-3- yl)methyl)-6-(6-(4-methylpiperazin- 1-yl)pyridin-3-yl)-1H-indazole-4- carboxamide | 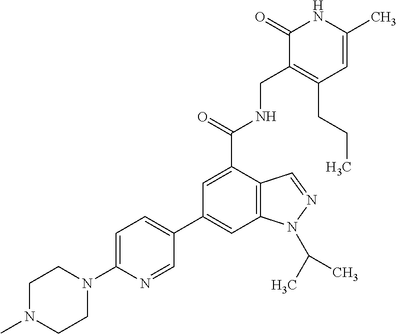 |
| N-[(4,6-Dimethyl-2-oxo-1,2- dihydro-3-pyridinyl)methyl]-3- methyl-1-(1-methylethyl)-6-[6-(4- methyl-1-piperazinyl)-3-pyridinyl]- 1H-indole-4-carboxamide-d8 | 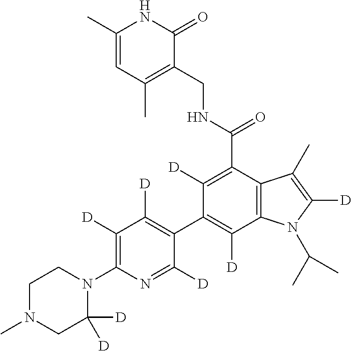 [SMILES: [2H]c1nc(N2CCN(C)CC2([2H])[2H])c([2H])c([2H])c1-c1c([2H])c(C(=O)NCc2c(C)cc(C)[nH]c2=O)c2c(C)c([2H])n(C(C)C)c2c1[2H]] |

### Treatment Methods

In one embodiment, the present invention provides methods for treatment, inhibition, prevention, or reduction of a cardiovascular using an inhibitor of EZH2 of the invention. In one embodiment, the inhibitor is a small molecular compound selected from Table 1. In one embodiment, the inhibitor is a siRNA comprising a sequence that forms a complex with or is complementary to a region in the EZH2 mRNA. In one embodiment, the siRNA comprises a sequence that forms a complex with or is complementary to a region in the EZH2 mRNA having a sequence selected from SEQ ID NO:1, SEQ ID NO:2 or SEQ ID NO:3. Treatment of cardiovascular diseases and conditions, which is generally understood to refer to diseases, conditions, or disorders involving the heart or blood vessels. Non-limiting examples of cardiovascular diseases include but are not limited to atherosclerosis, atherosclerosis-associated diseases, peripheral arterial occlusive disease, congestive heart failure, hypertension, cerebrovascular disease, dyslipidemia, and vasospastic disorders, including Raynaud's disease.

In one embodiment, the invention provides methods for improving endothelial function including endothelial cellular repair or replacement, and improving blood flow yielding enhanced oxygenation by inhibiting EZH2 in an endothelial cell.

In one embodiment, the present invention provides the use of an inhibitor of EZH2 of the invention or a pharmaceutically acceptable salt thereof for the preparation of a pharmaceutical composition for the treatment or prevention of an early cardiac or early cardiovascular disease in a patient in need thereof. By an early cardiac or early cardiovascular disease is meant a stage of disease prior to stroke or myocardial infarct.

In one embodiment the early cardiac or early cardiovascular disease is selected from the group consisting of left ventricular hypertrophy, coronary artery disease, essential hypertension, acute hypertensive emergency, cardiomyopathy, heart insufficiency, exercise intolerance, chronic heart failure, arrhythmia, cardiac dysrhythmia, syncopy, mild chronic heart failure, angina pectoris, cardiac bypass reocclusion, intermittent claudication (atheroschlerosis oblitterens), diastolic dysfunction and systolic dysfuntion.

The methods and compositions of the present invention may be used to treat advanced class 3B and class 4 heart failure, acute decompensated heart failure, cardio renal syndrome defined by biventricular failure, decreased glomerular filtration rate and systemic congestion, as well as acute coronary syndromes and microvascular angina. These compositions and methods have the possibility to reduce symptoms, reduce hospitalizations and increase the quality of life for patients with these conditions. In preferred embodiments the compositions are administered by continuous intravenous infusion which may be combined with standard therapies.

In another embodiment the patient suffers from a disease selected from the group consisting of myocardial infarct, acute coronary syndrome, unstable angina, non-Q-wave cardiac necrosis, Q-wave myocardial infarct and morbidity after stroke.

In another embodiment, the patient having the cardiovascular disease is a diabetic patient. In yet another embodiment, the patient having the cardiovascular disease is a non-diabetic patient.

The methods and compositions of the present invention may be used to provide acute cardioprotective effects, such as reducing the incidence of sudden death due to arrhythmias or contractile failure in a subject with an acute occlusion of a coronary artery (myocardial infarction); reducing damage occurring during reperfusion of the heart muscle after ischemia (‘hypoxia-reperfusion injury’ or ‘ischemia-reperfusion injury’); reducing the amount of cardiac muscle that is damaged or reducing the severity of damage to the heart muscle caused by an acute coronary artery occlusion (often referred to as ‘reducing infarct size’) Chronic cardioprotective effects include, but are not limited to, reducing pathologic remodeling of the cardiac chambers, including chamber dilation, consequent to an acute coronary artery occlusion; reducing apoptosis in cardiac muscle consequent to an acute coronary artery occlusion; reducing the impairment of contractility of cardiac muscle consequent to an acute coronary occlusion; and reducing long-term mortality in subjects have suffered damage to the heart muscle caused by an acute coronary occlusion.

Acute and/or chronic cardioprotective effects can be desirable in subjects with chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have an inotropic effect, increasing the strength of contraction in a failing heart. Acute and chronic inotropic effects may be desirable in acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention may also have an anti-arrhythmic effect. This effect can be acute or chronic, and can include effects that are attributable to prevention and/or reduction of injury to the heart muscle. Examples of anti-arrthymic effects include, but are not limited to, reducing the incidence and altering the rates of cardiac arrhythmias (including but not limited to atrial fibrillation, other supraventricular arrhythmias, ventricular tachycardia and ventricular fibrillation) following coronary occlusion.

The methods and compositions of the present invention may also have an anti-hypertrophic effect. Anti-hypertrophic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have lusitropic effects, improving the relaxation of the heart muscle during diastole. Lusitropic effects can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The methods and compositions of the present invention can also have anti-arrhythmic effects of benefit in the treatment of disorders of the heart rhythm, examples of which include but are not limited to atrial fibrillation, ventricular tachycardia and ventricular fibrillation. These effects, which can include reductions in the incidence and rate of the arrhythmias, can be desirable in subjects with acute coronary artery disease, chronic coronary artery disease (in which blood flow to the heart muscle is compromised without an acute coronary occlusion, also referred to as ischemic heart disease), myocarditis, idiopathic dilated cardiomyopathy, hypertrophic cardiomyopathy, restrictive cardiomyopathy, infiltrative cardiomyopathy, valvular heart disease, adult congenital heart disease, toxic cardiomyopathy (including but not limited to doxorubicin-induced cardiomyopathy), hypertensive cardiomyopathy, cardiomyopathy associated with endocrine disease, including diabetes, cardiomyopathy associated with connective tissue disease, cor pulmonale, pulmonary arterial hypertension, pulmonary embolism.

The patient treated using the methods and compositions of the present invention can also be at an increased risk of developing heart disease. This can include (but is not limited to) individuals with hypertension (systemic or pulmonary), obesity, endocrine disease (including diabetes, thyroid disease, adrenal disease, dysregulation of homocysteine metabolism), iron storage disease, amyolidosis, renal disease, connective tissue disease, infectious diseases, thromboembolic disease, immune diseases, hematologic diseases.

Provided herein are methods of increasing or enhancing the chances of survival of a subject with heart disease, comprising administering to a subject in need thereof an effective amount of an inhibitor of EZH2 of the invention, thereby increasing or enhancing the chances of survival of the subject treated by a certain period of time, for example, by at least 10 days, 1 month, 3 months, 6 months, 1 year, 1.5 years, 2 years, 3 years, 4 years, 5 years, 8 years, or 10 years. The increase in survival of a subject can be defined, for example, as the increase in survival of a preclinical animal model by a certain period of time, for example, by at least 10 days, 1 month, 3 months, 6 months, or 1 year, or at least 2 times, 3 times, 4 times, 5 times, 8 times, or 10 times, more than a control animal model (that has the same type of disease) without the treatment with the inventive method. Optionally, the increase in survival of a mammal can also be defined, for example, as the increase in survival of a subject with heart disease by a certain period of time, for example, by at least 10 days, 1 month, 3 months, 6 months, 1 year, 1.5 years, 2 years, 3 years, 4 years, 5 years, 8 years, or 10 years more than a subject with the same type of heart disease but without the treatment with the inventive method. The control subject may be on a placebo or treated with supportive standard care such as chemical therapy, biologics and/or radiation that do not include the inventive method as a part of the therapy.

### Pharmaceuticals

An inhibitor of EZH2 of the invention can be formulated and administered to a subject, are now described. The invention encompasses the preparation and use of pharmaceutical compositions comprising a composition useful for the treatment of a cardiovascular disease or disorder. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. As used herein, the term “pharmaceutically-acceptable carrier” means a chemical composition with which an appropriate peptide composition, may be combined and which, following the combination, can be used to administer the appropriate peptide composition to a subject.

The present invention includes pharmaceutical compositions comprising an inhibitor of EZH2. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, epidural, intracerebral, intracerebroventricular, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

### Combination Therapy

The preventive or therapeutic compositions of the present invention can also be used in combination with conventional therapeutics of heart failure such as diuretics, inotropes, coronary vasodilators and beta blockers or conventional therapeutics of circulatory diseases such as hypertension (e.g. angiotensin converting enzyme (ACE) inhibitors, angiotensin receptor blockers (ARBs) and/or calcium channel blockers), either simultaneously or at different times. Diuretics are generally used for relief of congestive symptoms and help the kidneys rid the body of excess fluid, thereby reducing blood volume and the heart's workload. Diuretics can include, but are not limited to loop diuretics (e.g. furosemide, bumetanide); thiazide diuretics (e.g. hydrochlorothiazide, chlorthalidone, chlorthiazide); potassium-sparing diuretics (e.g. amiloride); spironolactone and eplerenone. Inotropes, such as a cardiac glycoside, a beta-adrenergic agonist or a phosphodiesterase inhibitor, strengthen the heart's pumping action in patients with low cardiac output; inotropes can include but are not limited to digoxin, dobutamine, milrinone, istaroxime, omecamtiv mecarbil. Vasodilators, cause the peripheral arteries to dilate, making it easier for blood to flow; examples of vasodilators include, but are not limited, nitroglycerin, nitorprusside, and neseritide. Activation of neurohormonal systems that include the renin-andiotensin-aldosterone system (RAAS) and the sympathetic nervous system also contribute to the pathophysiology of heart failure. Drugs that inhibit activation of RAAS fall into three major categories: ACE inhibitors (including but not limited to ramipril, enalapril, and captopril), ARBs (including but not limited to valsarten, candesarten, irbesarten and losarten), and aldosterone receptor blockers (e.g., spironolactone and eplerenone.) Beta blockers counter the effects of activation of the sympathetic nervous system and slow the heart rate by blocking the effects of adrenalin; beta blockers include, but are not limited to carvedilol, metoprolol, bisoprolol, atenolol, propranolol, timolol and bucindolol.

### Kits

The present invention also pertains to kits useful in the methods of the invention. Such kits comprise various combinations of components useful in any of the methods described elsewhere herein, including for example, an inhibitor of EZH2, materials for quantitatively analyzing EZH2 or downstream effectors, materials for assessing the activity of EZH2 or downstream effectors, materials for assessing the treatment of a disease or disorder by administrating of an inhibitor of EZH2, and an instructional material.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Inhibiting EZH2 Stimulates Athero-Protective and Anti-Inflammatory Gene Expression in Endothelial Cells

Cardiovascular disease is the leading cause of death in the world. A sedentary (inactive) lifestyle is one of the top risk factors for heart disease. In contrast, regular exercise has many benefits including strengthen our heart and cardiovascular system and lower blood pressure. One major mechanism by which exercise improves cardiovascular function is that fluid shear stress generated by flowing blood that can trigger many signal pathways leading to gene expression and activation in vascular endothelial cells lining on the inner surface of blood vessels. Epigenetics, the study of inheritable changes in gene expression or cellular phenotype that not caused by the alternation of DNA sequence, has many potential medical applications and drug discovery.

The results presented herein demonstrate that inhibition of EZH2 by GSK126, an EZH2 inhibitor, stimulated gene expression in vascular endothelial cells including Kruppel-like factors 2 (KLF2) and endothelial nitric oxide synthase (eNOS), an enzyme that generates the vasoprotective molecule nitric oxide. Both KLF2 and eNOS play important roles in preventing endothelial dysfunction and vascular inflammation, two hallmarks of cardiovascular diseases. Collectively, the findings suggest that the inhibition of EZH2 by GSK126 or other compounds is an effective therapeutic strategy to prevent and treat cardiovascular diseases.

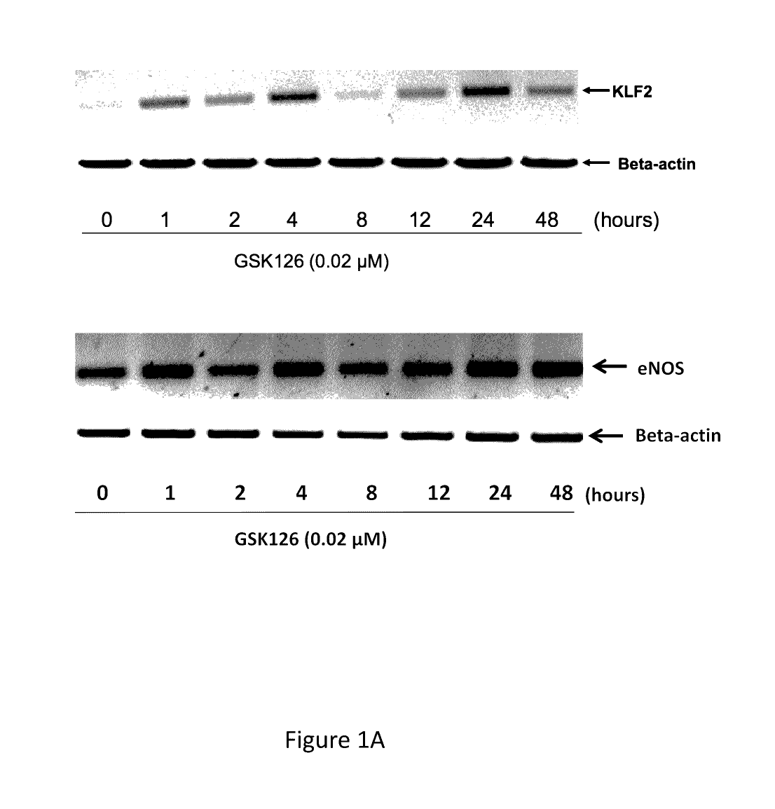

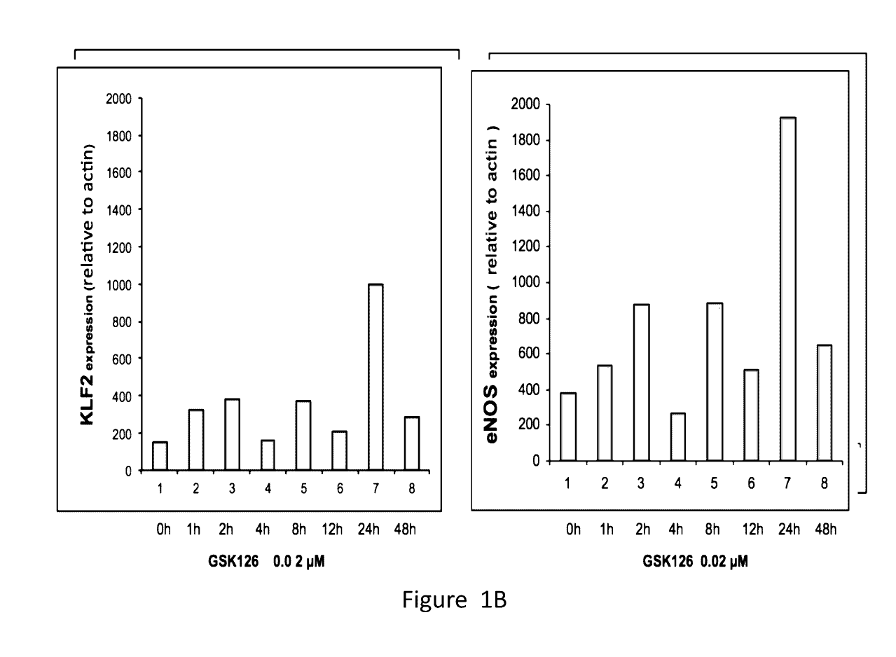

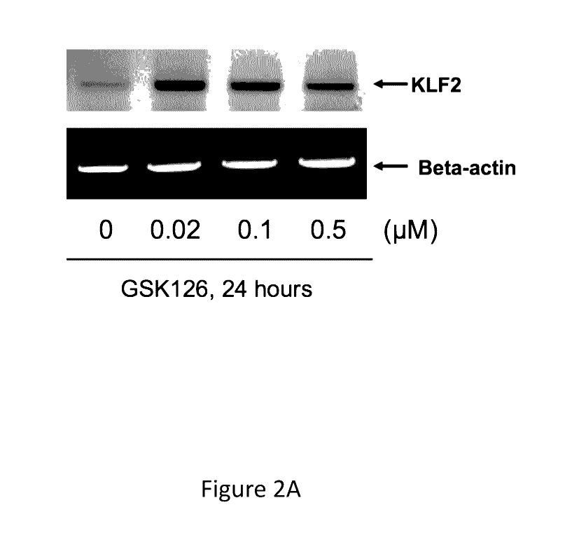

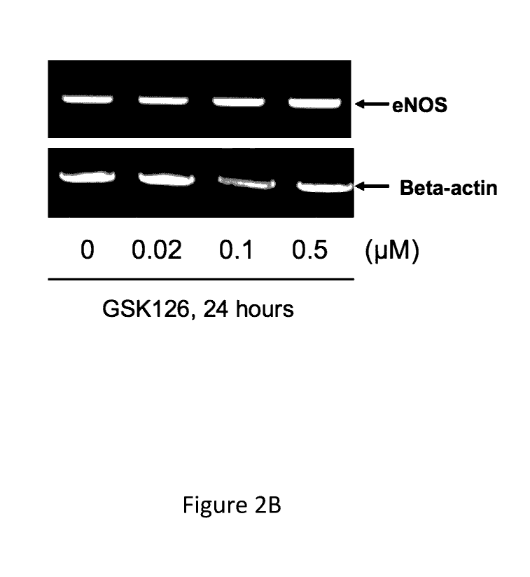

Experiments were designed to determine the effects of the EZH2 inhibitor GSK126 on expression of genes KLF2 and eNOS in endothelial cells. Histone methylation is an important epigenetic modification. Histone 3 lysine 27 (H3K27) is correlated with transcriptional repression. EZH2, a histone methytransferase, is critical for the epigenetic maintenance of the H3K27me3 repressive chromatin mark. To determine whether the alternation of histone methylation affects gene expression in endothelial cells, experiments were designed to assess the effects of an EZH2 inhibitor GSK126. To this end, human umbilical vein endothelial cells (HUVECs) were treated with 0.02 μM GSK126 for different times, and the cells were collected for the measurement of gene expression level using the assays of a semi-quantitative reverse transcription polymerase chain reaction (RT-PCR) and a quantitative real-time PCR (q-PCR). As shown in FIG. 1, the treatment of GSK126 increased the level of KLF2 and eNOS mRNA in HUVECs in a time-dependent manner. Similarly, when HUVECs were treated GSK126 for 24 hours at the different concentrations, it was observed that GSK126 dose-dependently increased KLF2 and eNOS expression in endothelial cells (FIG. 2). Collectively, these results demonstrate that inhibiting EZH2 by GSK126 stimulates atheroprotective gene expression in endothelial cells.

### Example 2

### Inhibiting EZH2 as a Therapeutic Strategy to Prevent Atherosclerosis-Associated Cardiovascular Disease

The results presented herein demonstrate that the inhibition of EZH2 enhances expression of atheroprotective genes in vascular endothelial cells and attenuates the formation of experimental atherosclerosis. In cultured human endothelial cells, knockdown of EZH2 by small interference RNA increased expression of Kruppel-like factor 2 (KLF2) and endothelial nitric oxide synthase (eNOS) genes, two key molecules that regulate endothelial homeostasis and inflammation. Total deletion of EZH2 in mice results in early embryonic lethality. To determine the effect of heterozygous EZH2 deficiency (EZH2^{+/-}) on atherosclerosis, apolipoprotein E deficient (ApoE^{-/-}); EZH2^{+/-} mice were generated and fed western diet. Compared to ApoE^{-/-}; EZH2^{+/+} control mice, ApoE^{-/-}; EZH2^{+/-} developed less atherosclerotic lesions. Therefore, inhibiting EZH2 provides a therapeutic strategy to prevent atherosclerosis-associated cardiovascular disease.

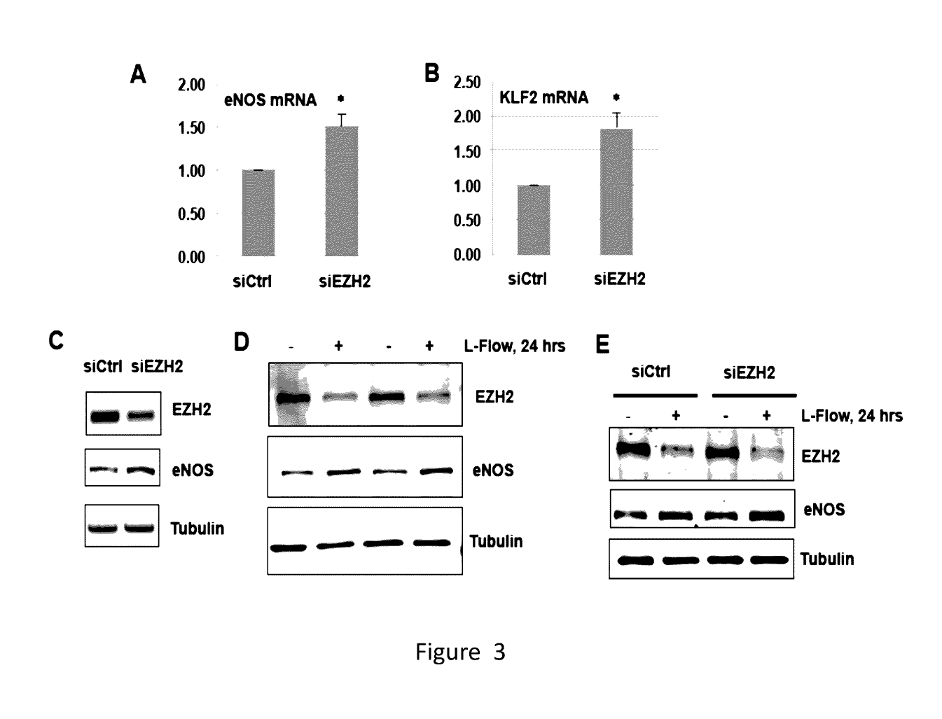

Experiments were designed to evaluate the role of EZH2 in the regulation of vascular endothelial homeostasis. Experiments were designed to study the effect of knockdown EZH2 by small interference RNA (siRNA) on expression of endothelial genes Kruppel-like factor 2 (KLF2) and endothelial nitric oxide synthase (eNOS), two important atheroprotective molecules. As shown in FIG. 3A-3C, the treatment of EZH2 siRNAs that are complementary to the region in the EZH2 mRNA represented by SEQ ID NOs: 1, 2 & 3 decreased EZH2 mRNA and protein expression in cultured human endothelial cells. EZH2 knockdown inhibited KFL2 and eNOS mRNA expression as well as eNOS protein expression in ECs (FIG. 3A-3C). It was also observed that laminar flow, the atheroprotective force generated by flowing blood especially during exercise, decreased EZH2 protein expression in cultured human endothelial cells (FIG. 3D). The combination of the treatment of EZH2 siRNA and the exposure of laminar flow has synergistic effects on eNOS expression in human endothelial cells (FIG. 3E).

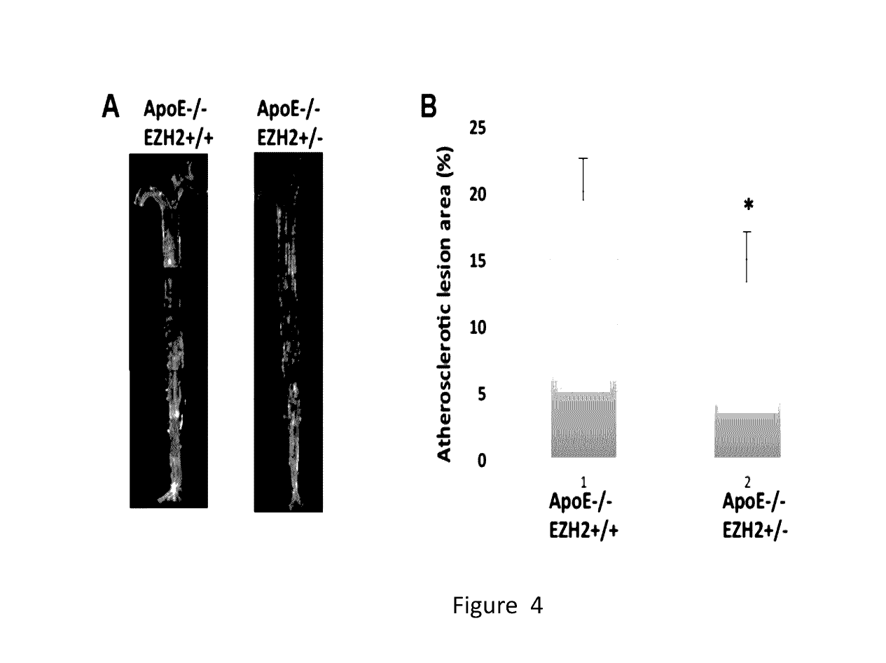

Experiments were also designed to determine the functional consequences of EZH2 deficiency. EZh2 knockout mice were generated by breeding Ezh2^{F/F} mutant mice (Jackson laboratory) possessing loxP sites flanking exons 14-15 of the zeste homolog 2 (Ezh2) gene with mouse EIIa-cre line (Jackson laboratory) that carries a cre transgene under the control of the adenovirus Ella promoter that targets expression of Cre recombinase to the early mouse embryo. Homozygous mice with total deletion of EZH2 gene were embryonic lethal. However, the heterozygous EZH2 (EZH2^{+/-}) mice in which only one allele of EZH2 is deleted are viable, fertile, normal in size and do not display any gross physical or behavioral abnormalities. To investigate the role of EZH2 in regulation of atherosclerosis, EZH2^{+/-} mice were breed with ApoE^{-/-} mice on C57BL/6J background (Jackson laboratory) to generate ApoE^{-/-}, EZH2^{+/-} mice and their littermate ApoE^{-/-}, EZH2^{+/+} control mice. For atherosclerosis study, both ApoE^{-/-}, EZH2^{+/-} and ApoE^{-/-}, EZH2+/+ mice were fed on Western diet (Rodent Western Diet #D12079B, Research Diet, New Brunswick, N.J.) for 8 weeks beginning at 7 weeks of age. The mice were housed under a 12-h light/dark cycle in specific-pathogen free facility. Compared with vehicle control group, ApoE^{-/-}, EZH2^{+/-} mice developed less atherosclerotic lesion formation in the en face prepared aorta (FIGS. 4A and 4B). These data suggest that haploinsufficiency of EZH2 is associated with reduced atherosclerotic lesion formation.

Experiments were also designed to use vascular endothelial cell-specific EZH2 knockout mice in the background of ApoE deficiency to evaluate the functional role of EZH2 in regulating endothelial function and atherosclerosis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating a cardiovascular disease in a subject comprising administering to a subject an effective amount of a compound selected from the group consisting of (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide, N-((4,6-dimethyl-2-oxo-1,2-dihydropyridin-3-yl)methyl)-1-isopropyl-3-methyl-6-(6-(4-methylpiperazin-1-yl)pyridin-3-yl)-1H-indole-4-carboxamide, N-((4,6-dimethyl-2-oxo-1,2-dihydropyridin-3-yl)methyl)-6-(6-(hydroxymethyl)pyridin-3-yl)-1-isopropyl-3-methyl-1H-indole-4-carboxamide, N-((4,6-dimethyl-2-oxo-1,2-dihydropyridin-3-yl)methyl)-1-isopropyl-3-methyl-6-(oxetan-3-yl)-1H-indole-4-carboxamide, N-((4,6-dimethyl-2-oxo-1,2-dihydropyridin-3-yl)methyl)-1-isopropyl-3-methyl-6-(4-methylpiperazine-1-carboxamido)-1H-indole-4-carboxamide, N-((4,6-dimethyl-2-oxo-1,2-dihydropyridin-3-yl)methyl)-6-((3-(dimethylamino)propyl)thio)-1-isopropyl-3-methyl-1H-indole-4-carboxamide, N-((4,6-dimethyl-2-oxo-1,2-dihydropyridin-3-yl)methyl)-6-((3-(dimethylamino)propyl)thio)-1-isopropyl-3-methyl-1H-indole-4-carboxamide, 6-(3-hydroxy-3-methylbut-1-yn-1-yl)-1-isopropyl-3-methyl-N-((6-methyl-2-oxo-4-propyl-1,2-dihydropyridin-3-yl)methyl)-1H-indole-4-carboxamide, 6-(cyclopropylethynyl)-1-isopropyl-3-methyl-N-((6-methyl-2-oxo-4-propyl-1,2-dihydropyridin-3-yl)methyl)-1H-indole-4-carboxamide, 1-cyclopentyl-N-((4,6-dimethyl-2-oxo-1,2-dihydropyridin-3-yl)methyl)-6-(morpholinomethyl)-1H-indazole-4-carboxamide, N-((4,6-dimethyl-2-oxo-1,2-dihydropyridin-3-yl)methyl)-3-(ethyl(tetrahydro-2H-pyran-4-yl)amino)-2-methyl-5-(morpholinomethyl)benzamide, (1S,2R,5R)-5-(4-amino-1H-imidazo [4,5-c]pyridin-1-yl)-3-(hydroxymethyl)cyclopent-3-ene-1,2-diol, 1-isopropyl-N-((6-methyl-2-oxo-4-propyl-1,2-dihydropyridin-3-yl)methyl)-6-(6-(4-methylpiperazin-1-yl)pyridin-3-yl)-1H-indazole-4-carboxamide, and N-[(4,6-Dimethyl-2-oxo-1,2-dihydro-3-pyridinyl)methyl]-3-methyl-1-(1-methylethyl)-6-[6-(4-methyl-1-piperazinyl)-3-pyridinyl]-1H-indole-4-carboxamide-d8.

2. The method of claim 1, the compound is (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1 H-indole-4-carboxamide.

3. The method of claim 1, the compound is 1-isopropyl-N-((6-methyl-2-oxo-4-propyl-1,2-dihydropyridin-3-yl)methyl)-6-(6-(4-methylpiperazin-1-yl)pyridin-3-yl)-1H-indazole-4-carboxamide.

4. A method for treating a cardiovascular disease in a subject, the method comprising administering to a subject in need thereof an effective amount of a siRNA that forms a complex with a region in EZH2 mRNA.

5. The method of claim 4, wherein the siRNA comprises a sequence complementary to a region in EZH2 mRNA.

6. The method of claim 5, wherein the siRNA comprises a sequence that is complementary to a region having a sequence selected from the group consisting of SEQ ID NOs:1, 2 and 3.

7. The method of claim 1, wherein the cardiovascular disease is selected from the group consisting of coronary artery disease, hypertension, heart failure, diabetic cardiovascular complications, atherosclerosis, coronary heart disease, angina, stroke, ischemia and myocardial infarction, and any combination thereof.

8. The method of claim 1 further comprising administering a second agent to the subject.

9. The method of claim 8, wherein the second agent is selected from the group consisting of ACE inhibitors, ARB's, adrenergic blockers, adrenergic agonists, agents for pheochromocytoma, anti-arrhythmics, antiplatelet agents, anticoagulants, antihypertensives, antilipemic agents, antidiabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, renin inhibitors, vasodilators, vasopressors, AGE crosslink breakers, AGE formation inhibitors, and any combinations thereof.

10. The method of claim 4, wherein the cardiovascular disease is selected from the group consisting of coronary artery disease, hypertension, heart failure, diabetic cardiovascular complications, atherosclerosis, coronary heart disease, angina, stroke, ischemia and myocardial infarction, and any combination thereof.

11. The method of claim 4 further comprising administering a second agent to the subject.

12. The method of claim 11, wherein the second agent is selected from the group consisting of ACE inhibitors, ARB's, adrenergic blockers, adrenergic agonists, agents for pheochromocytoma, anti-arrhythmics, antiplatelet agents, anticoagulants, antihypertensives, antilipemic agents, antidiabetics, anti-inflammatory agents, calcium channel blockers, CETP inhibitors, COX-2 inhibitors, direct thrombin inhibitors, diuretics, endothelin receptor antagonists, HMG Co-A reductase inhibitors, inotropic agents, renin inhibitors, vasodilators, vasopressors, AGE crosslink breakers, AGE formation inhibitors, and any combinations thereof.


SEQUENCE LISTING (3 sequences)

SEQ ID NO: 1 - RNA, 25 nt, Artificial sequence
  Chemically synthesized
aggauacagacagugauagggaagc

SEQ ID NO: 2 - RNA, 25 nt, Artificial sequence
  Chemically synthesized
ggcacuuacuaugacaauuucugug

SEQ ID NO: 3 - RNA, 25 nt, Artificial sequence
  Chemically synthesized
gcucuagacaacaaaccuuguggac